(19) United States
(12) Patent Application Publication
(10) Pub. No.: US 2025/0321198 A1
(43) Pub. Date: Oct. 16, 2025

(54) System for determination of a quantity of emissions of carbon dioxide resulting from the heating of an electrical conductor of an electrical cable by the Joule effect

(71) Applicant: NEXANS, Courbevoie (FR)

(72) Inventor: Raphaël GUFFOND, LYON (FR)

(21) Appl. No.: 19/039,080

(22) Filed: Jan. 28, 2025

(30) Foreign Application Priority Data
Feb. 21, 2024 (FR) 2401716

(51) Int. Cl. G01N 25/12

(52) U.S. Cl. CPC G01N 25/12

(57) ABSTRACT

A system (100) for determination of a quantity of emissions of carbon dioxide resulting from the heating of an electrical conductor of an electrical cable by the Joule effect includes an electrical cable (10) having at least one electrical conductor (12) and at least one layer of material surrounding the at least one conductor, and a measurement unit (110) associated with the electrical cable. The measurement unit has at least one temperature sensor (20) and a device (112) for measurement of the electrical intensity I_{cond} of an electrical current circulating in the electrical conductor. A calculation unit (120) is configured to communicate information with the measurement unit, the calculation unit being configured to determine the conductor temperature Θ_{cond} by means of the at least one temperature sensor. The calculation unit is further configured to determine a quantity of emissions of carbon dioxide resulting from the heating of the electrical conductor by the Joule effect as a function of the conductor temperature Θ_{cond} and the electrical intensity I_{cond} in the electrical conductor.

### RELATED APPLICATION

This application claims the benefit of priority from French Patent Application No. 24 01716, filed on Feb. 21, 2024, the entirety of which is incorporated by reference.

### TECHNICAL FIELD

The present invention concerns a system for determination of a quantity of emissions of carbon dioxide resulting from the heating of an electrical conductor of an electrical cable by the Joule effect.

The invention more specifically concerns a non-invasive determination system.

### TECHNOLOGICAL BACKGROUND

In an electrical distribution network, heating of the electrical conductor of an electrical cable is produced by the Joule effect when a current circulates in the electrical conductor. With this increase in temperature, the resistance of the electrical conductor increases, which leads to an increase in the power loss.

This annual power loss directly impacts the quantity of emissions of carbon dioxide (CO₂).

At present there exist no integrated or add-on solutions for determination of these emissions of carbon dioxide resulting from the heating of an electrical conductor of an electrical cable by the Joule effect.

Thus there exists a need for a system for determination of a quantity of emissions of carbon dioxide resulting from the heating of an electrical conductor of an electrical cable by the Joule effect directly integrated into an electrical cable or added thereto.

### SUMMARY OF THE INVENTION

To this end, the invention proposes a system for determination of a quantity of emissions of carbon dioxide resulting from the heating of an electrical conductor of an electrical cable by the Joule effect, said determination system comprising:
an electrical cable comprising at least one electrical conductor and at least one layer of material surrounding said at least one conductor,
a measurement unit associated with the electrical cable, said measurement unit comprising at least one temperature sensor and a device for measurement of the electrical intensity I_{cond} of an electrical current circulating in the electrical conductor,
a calculation unit configured to communicate information with the measurement unit, the calculation unit being configured to determine the conductor temperature Θ_{cond} by means of said at least one temperature sensor, said calculation unit being further configured to determine a quantity of emissions of carbon dioxide resulting from the heating of the electrical conductor by the Joule effect as a function of the conductor temperature Θ_{cond} and the electrical intensity I_{cond} in the electrical conductor.

The integration of a measurement module and a calculation unit configured to determine the conductor temperature Θ_{cond} and the electrical intensity I_{cond} within the same determination system makes it possible to determine the quantity of the emissions of CO₂ induced by the heating of the electrical conductor.

In accordance with one embodiment of the determination system, the calculation unit is configured to determine an electrical resistance R_{c} of the electrical conductor as a function of the conductor temperature Θ_{cond}.

This electrical resistance R_{c} is in particular determined as follows:

$${R}_{c} = {R}_{0} \times ( 1 + {α}_{20} \times ( {θ}_{cond} - 20 ) ) \times ( 1 + {y}_{s} + {y}_{p} )$$

In accordance with one embodiment of the determination system, the calculation unit is configured to determine a power loss PₒL as a function of the electrical intensity I_{cond} in the electrical conductor and the electrical resistance R_{c} of the electrical conductor, the calculation unit being configured to determine the quantity of emissions of carbon dioxide as a function of said power loss PₒL.

This power loss PₒL is in particular determined as follows:

$$PoL = Rc \times {I}_{cond}^{2}$$

In accordance with one embodiment of the determination system, the latter further comprises a measurement module mounted on the electrical cable, said measurement module comprising at least one of the following: said at least one temperature sensor and the device for measurement of the electrical intensity I_{cond}.

In accordance with one embodiment of the determination system, the measurement module further comprises the calculation unit.

In accordance with one embodiment of the determination system, the measurement module is configured to be removably mounted on the electrical cable.

In accordance with one embodiment of the determination system, the measurement module comprises a device for fixing it to the electrical cable.

In accordance with one embodiment of the determination system, said at least one temperature sensor is disposed on an external surface of said at least one layer of material to measure a peripheral temperature Θ_{b1} at the level of the external surface of said at least one layer of material, the calculation unit being configured to determine the conductor temperature Θ_{cond} as a function of the peripheral temperature Θ_{b1}.

The use of one or more temperature sensors external to the electrical cable enables determination of the conductor temperature Θ_{cond} in a non-invasive and non-destructive manner.

In accordance with one embodiment of the determination system, the latter further comprises:
an additional layer of material disposed around said at least one layer of material and covering said at least one temperature sensor,
at least one additional temperature sensor disposed on an external surface of said additional layer of material to measure an additional peripheral temperature Θ_{b2} at the level of the external surface of said at least one additional layer of material.

In accordance with one embodiment of the determination system, the calculation unit is configured to determine the conductor temperature Θ_{cond} as a function of the peripheral temperature Θ_{b1} and the additional peripheral temperature Θ_{b2}.

Said at least one layer of material of the electrical cable has a layer thermal resistance T₁.

The calculation unit may comprise a unit for determination of the conductor temperature Θ_{cond}. Thus the determination unit is configured to determine the conductor temperature Θ_{cond} as a function of the measured peripheral temperature Θ_{b1}, the layer thermal resistance T₁ and the thermal flux W_{c} generated by the circulation of an electrical current in the electrical conductor.

The conductor temperature Θ_{cond} is determined here by means of a physical model utilising the measured peripheral temperature Θ_{b1} and the layer thermal resistance T₁.

The use of this physical model makes it possible to dispense with the utilisation of a temperature internal to the electrical cable, i.e. measured via a component disposed in the proximity of the conductor, or more generally inside the external sheath of the electrical cable.

The physical model utilised enables estimation of the conductor temperature Θ_{cond} by means of the measured peripheral temperature Θ_{b1} and the layer thermal resistance T₁.

In accordance with one embodiment of the determination system, the latter further comprises:
at least one additional layer of material disposed around said at least one layer of material and covering said at least one temperature sensor, said additional layer of material having an additional thermal resistance T_{b},
at least additional temperature sensor disposed on an external surface of said additional layer of material to measure an additional peripheral temperature Θ_{b2} at the level of the external surface of said at least one additional layer of material.

In accordance with one embodiment of the determination system, said additional layer of material extends around said at least one layer of material over only a portion of the length of the electrical cable.

In accordance with one embodiment of the determination system, said at least one additional layer of material comprises:
a first additional layer portion having a first additional thermal resistance T_{b}, and
a second additional layer portion having a second additional thermal resistance T′_{b}, the first additional thermal resistance T_{b} and the second additional thermal resistance T′_{b} being different,
   and in which said at least one additional temperature sensor comprises:
at least one first additional temperature sensor disposed on an external surface of the first additional layer portion to measure a first additional peripheral temperature Θ_{b2},
at least one second additional temperature sensor disposed on an external surface of the second additional layer portion to measure a second additional peripheral temperature Θ′_{b2},
   the determination unit being configured to determine the conductor temperature Θ_{cond} further as a function of the first additional thermal resistance T_{b} and the second additional thermal resistance T′_{b} and the first additional peripheral temperature Θ_{b2} and the second additional peripheral temperature Θ′_{b2}.

In accordance with one embodiment of the determination system, said at least one first additional layer and said at least one second additional layer are disposed on different angular sectors around the electrical conductor.

Said at least one first additional layer and said at least one second additional layer may overlap at least partially.

In accordance with one embodiment of the determination system, said at least one first additional layer and said at least one second additional layer together form a layer extending continuously around the electrical conductor in a plane perpendicular to the longitudinal axis along which the electrical conductor extends.

In accordance with one embodiment of the determination system, said at least one temperature sensor comprises:
at least one first sensor disposed on the external surface of said at least one layer of material, between said external surface and the first additional layer portion, to measure a peripheral temperature Θ_{b1} at the level of the external surface of said at least one layer of material,
at least one second sensor disposed on the external surface of said at least one layer of material, between said external surface and the second additional layer portion, to measure a peripheral temperature θ′_{b1} at the level of the external surface of said at least one layer of material.

In accordance with one embodiment of the determination system, the determination unit is configured to determine the conductor temperature Θ_{cond} on the basis of the following equation:

$${θ}_{conducteur} = \frac{( {θ}_{b 1} {T}_{b} - {θ}_{b 1}^{′} {T}_{b}^{′} ) \times ( ( {θ}_{b 1} - {θ}_{b 2} ) )}{{T}_{b} ( {θ}_{b 1}^{′} - {θ}_{b 2}^{′} ) - {T}_{b}^{′} ( {θ}_{b 1} - {θ}_{b 2} )}$$

T₁ being the thermal resistance of said at least one layer of material,
T_{b} being the first additional thermal resistance of the first additional layer of material,
T′_{b} being the second additional thermal resistance of the second additional layer of material,
Θ_{b1} is the peripheral temperature measured by said at least one first temperature sensor,
Θ_{b2} is the additional peripheral temperature measured by said at least one first additional temperature sensor,
Θ′_{b1} is the peripheral temperature measured by said at least one second temperature sensor,
Θ′_{b2} is the additional peripheral temperature measured by said at least one second additional temperature sensor.

In accordance with one embodiment of the determination system, the first and second additional layer portions are made of different materials.

In accordance with one embodiment of the determination system, the first and second additional layer portions have different thicknesses perpendicular to a longitudinal axis along which the electrical cable extends.

In accordance with one embodiment of the determination system, said at least one layer of material comprises an external sheath forming said external surface, said at least one temperature sensor being disposed on said external sheath.

In accordance with one embodiment of the determination system, the determination unit is configured to determine a conductor temperature Θ_{cond} as a function of the measured peripheral temperature Θ_{b1} and the layer thermal resistance T₁ and the thermal flux W_{c} generated by the circulation of an electrical current in the electrical conductor.

In accordance with one embodiment of the determination system, the determination unit is configured to determine the conductor temperature Θ_{cond} on the basis of the following equation:

$${θ}_{cond} = {θ}_{b 1} + {W}_{c} * {T}_{1}$$

The determination unit may be disposed in the proximity of the electrical cable or at a distance from the latter.

In accordance with one embodiment of the determination system, said at least one layer of material comprises an external sheath forming said external surface, said at least one temperature sensor being disposed on said external sheath.

In accordance with one embodiment of the determination system, the latter further comprises a device for measuring the electrical intensity I_{cond} of an electrical current circulating in said at least one electrical conductor, the determination unit being configured to determine said conductor temperature Θ_{cond} as a further function of that electrical intensity I_{cond}.

In accordance with one embodiment of the determination system, the measuring device is a non-invasive device, notably of the Rogowski coil type.

In accordance with one embodiment of the determination system, the conductor temperature Θ_{cond} is determined as follows:

$${θ}_{cond} = {θ}_{b 1} + {R}_{c} * {I}_{cond}^{2} * {T}_{1}$$

R_{c} being the electrical resistance of the conductor.

In accordance with one embodiment of the determination system, the latter further comprises:
an additional layer of material disposed around said at least one layer of material and covering said at least one temperature sensor, said additional layer of material having an additional thermal resistance T_{b},
at least one additional temperature sensor disposed on an external surface of said additional layer of material to measure an additional peripheral temperature Θ_{b2} at the level of the external surface of said at least one additional layer of material.

In accordance with one embodiment of the determination system, said additional layer of material extends around said at least one layer of material over only a portion of the length of the electrical cable.

In accordance with one embodiment of the determination system, the determination unit is configured to determine the conductor temperature Θ_{cond} on the basis of the following equation:

$${θ}_{cond} = {θ}_{b 1} + \frac{( {θ}_{b 1} - {θ}_{b 2} )}{{T}_{b}} \times {T}_{1}$$

where
Θ_{b1} is the peripheral temperature measured by said at least one temperature sensor,
Θ_{b2} is the additional peripheral temperature measured by said at least one additional temperature sensor,
T_{b} being the additional thermal resistance of the additional layer of material,
T₁ being the thermal resistance of said at least one layer of material.

In accordance with one embodiment of the determination system, the determination unit is configured to determine the conductor temperature Θ_{cond} as a further function of a heating W_{d} resulting from a dielectric loss in said at least one layer of material.

In accordance with one embodiment of the determination system, the determination unit is configured to determine the conductor temperature Θ_{cond} on the basis of the following equation:

$${θ}_{cond} = {θ}_{b 1} + {T}_{1} \times ( {W}_{c} - \frac{{W}_{d}}{2} )$$

W_{d} being the heating resulting from a dielectric loss in said at least one layer of material.

In accordance with one embodiment of the determination system, the determination unit is configured to determine the conductor temperature Θ_{cond} on the basis of the following equation:

$${θ}_{cond} = {θ}_{b 1} + {T}_{1} \times ( \frac{{θ}_{b 1} - {θ}_{b 2}}{{T}_{b}} - \frac{{W}_{d}}{2} )$$

W_{d} being the heating resulting from a dielectric loss in said at least one layer of material.

In accordance with one embodiment of the determination system, said at least one additional layer of material and said at least one additional temperature sensor are carried by the measurement module.

This measurement module is preferably removable from the electrical cable in such a manner as to carry out a point and localised measurement on the electrical cable. This unit is for example a measurement accessory.

The measurement module preferably has a dimension along the longitudinal axis of the electrical cable such that it extends over only a portion of the length of the electrical cable.

### BRIEF DESCRIPTION OF THE FIGURES

The following description given with reference to the appended drawings, provided by way of non-limiting example, will clearly explain in what the invention consists and how it may be carried out. In the appended figures:

FIG. 1 represents a perspective view of a determination system comprising an electrical cable, a measurement module, a calculation unit for determining a quantity of emissions of carbon dioxide resulting from the heating of an electrical conductor of the electrical cable;

FIG. 2 represents a perspective view of one embodiment of the determination system from FIG. 1 comprising a measurement module mounted on the electrical cable; the measurement module comprises in particular the measurement unit;

FIG. 3 represents a view in section of the electrical cable in a first configuration comprising an electrical conductor and at least one layer of material;

FIG. 4 represents a view in section of the electrical cable from FIG. 3 comprising a plurality of sensors on an external surface of said at least one layer of material;

FIG. 5 represents a diagram of a first model of the electrical cable in a first determination mode comprising determination of the electrical intensity of the electrical current circulating in the electrical conductor;

FIG. 6 represents a view in section of the electrical cable from FIG. 3 in a second determination mode in which the determination system comprises an additional layer of material around the electrical cable and a plurality of additional sensors disposed on an external surface of that additional layer of material;

FIG. 7 represents a diagram of a second model of the electrical cable in the second determination mode;

FIG. 8 represents a third model of the electrical cable in which the dielectric losses in said at least one layer of material are taken into account in the first determination mode;

FIG. 9 represents a fourth model of the electrical cable in which the dielectric losses in said at least one layer of material are taken into account in the second determination mode;

FIG. 10 represents a view in section of the electrical cable in a second configuration comprising an electrical conductor, one or more layers of material surrounding the electrical conductor and a screen surrounding said layer(s) of material;

FIG. 11 represents a fifth model of the electrical cable in which the dielectric losses in said at least one layer of material are taken into account as well as the dielectric losses in the screen;

FIG. 12 represents a sixth model of the electrical cable for determining the conductor temperature Θ_{cond} in a manner independent of the surrounding environment;

FIG. 13 represents a view in section of the electrical cable from FIG. 6 in a second determination mode in which the determination system comprises an additional layer of material formed of first and second additional layer portions and a plurality of additional sensors disposed on an external surface of each of the first and second additional layer of material portions;

FIG. 14 represents a diagram of a seventh model of the electrical cable utilising the first additional layer portion from FIG. 13;

FIG. 15 represents a diagram of the seventh model of the electrical cable utilising the second additional layer portion from FIG. 13;

FIG. 16 represents an equation for determining the conductor temperature using the seventh model;

FIG. 17 represents a diagram of an eighth model of the electrical cable utilising the first additional layer portion from FIG. 13;

FIG. 18 represents a diagram of the eighth model of the electrical cable utilising the second additional layer portion from FIG. 13;

FIG. 19 represents an equation for determining the conductor temperature utilising the eighth model.

### DESCRIPTION OF EMBODIMENT(S)

For reasons of clarity, the same references designating the same elements according to the prior art and according to the invention are used for all the figures.

The concept of the invention is described more completely hereinafter with reference to the appended drawings, in which embodiments of the concept of the invention are shown. In the drawings, the size and the relative sizes of the elements may be exaggerated for reasons of clarity. Similar numbers refer to similar elements in all the drawings. However, this concept of the invention may be executed in numerous different forms and should not be interpreted as being limited to the embodiments disclosed here. Rather than that, these embodiments are proposed so that this description is complete and communicates the extent of the concept of the invention to persons skilled in the art.

Any reference throughout the specification to “an embodiment” signifies that a functionality, a structure or a particular feature described with reference to one embodiment is included in at least one embodiment of the present invention. Thus the occurrence of the expression “in one embodiment” at various places throughout the specification does not necessarily refer to the same embodiment. Furthermore, the functionalities, the structures or the particular features may be combined in any appropriate manner in one or more embodiments. Furthermore, the term “comprising” does not excluded other elements or steps.

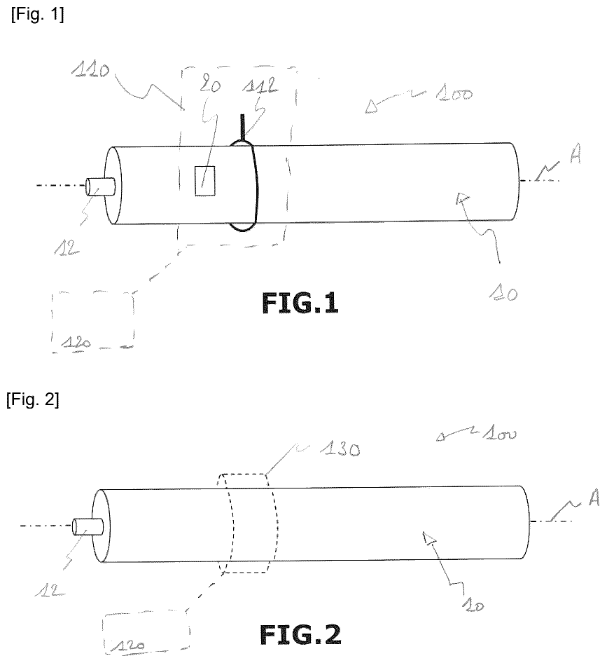

A system 100 for determination of a quantity of emissions of carbon dioxide resulting from the heating of an electrical conductor of an electrical cable by the Joule effect is depicted in FIG. 1.

This determination system 100 comprises an electrical cable 10 comprising at least one electrical conductor 12 and at least one layer of material surrounding said at least one conductor. This layer of material is for example an insulating layer. The electrical cable 10 extends along a longitudinal axis A.

This determination system 100 comprises a measurement unit 110 associated with the electrical cable 10. The measurement unit comprises at least one temperature sensor 20 and a device 112 for measuring the electrical intensity I_{cond} Of an electrical current circulating in the electrical conductor 12.

The measurement device 112 is preferably a non-invasive device, in particular of the Rogowski coil type.

The determination system 100 further comprises a calculation unit 120 configured to communicate information with the measurement unit 110.

The calculation unit 120 is configured to determine the conductor temperature Θ_{cond} by means of said at least one temperature sensor.

Said at least one temperature sensor 20 is preferably disposed outside the electrical cable, i.e. on an external surface of this electrical cable 10. In this configuration, the conductor temperature Θ_{cond} is determined by means of a physical model described hereinafter with reference to FIGS. 4 to 12.

In a variant compatible with the invention, said at least one temperature sensor may be disposed inside the electrical cable 10. In this variant, the conductor temperature Θ_{cond} is measured directly in the proximity of the electrical conductor 12.

The calculation unit 120 is further configured to determine a quantity of emissions of carbon dioxide resulting from the heating of the electrical conductor by the Joule effect as a function of the conductor temperature Θ_{cond} and the electrical intensity I_{cond} in the electrical conductor.

Referring to FIG. 2, the determination system 100 may comprise a measurement module 130 in which are accommodated one or more of the following: said at least one temperature sensor 20 and the measurement device 112.

The entire measurement unit 110 is preferably carried by the measurement module 130.

The calculation unit 120 is configured to be in communication with the measurement unit 110. The calculation unit 120 may be dissociated from the measurement module 130 as depicted in FIG. 2 or integrated into this measurement module 130.

This measurement module 130 is for example mounted in such a manner as to be removable from the electrical cable 10 in order to produce a point and localised measurement on the electrical cable 10. This measurement module 130 is for example a portable measurement accessory.

The measurement module 130 has a dimension along the longitudinal axis A such that it extends over only a portion of the length of the electrical cable 10. The measurement module 130 preferably extends around the electrical cable 10, i.e. around the longitudinal axis A.

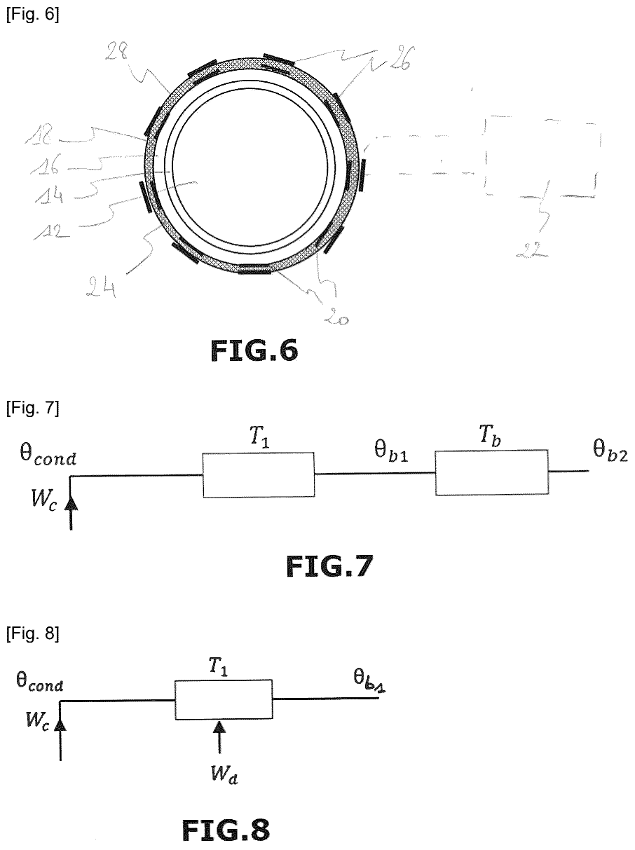

The measurement module 130 may equally comprise an additional structure described hereinafter, notably with reference to FIGS. 6 and 10.

For the determination of the emissions of CO₂, the calculation unit 120 is configured to determine an electrical resistance R_{c} of the electrical conductor as a function of the conductor temperature Θ_{cond}.

This electrical resistance R_{c} is notably determined as follows:

$${R}_{c} = {R}_{0} \times ( 1 + {α}_{20} \times ( {θ}_{cond} - 20 ) ) \times ( 1 + {y}_{s} + {y}_{p} )$$

with
R₀ being the direct current resistance of the conductor at 20° C., in Ohms,
Yₛ being the skin effect factor, no units,
Yₚ being the proximity effect factor, no units,
α₂₀ being the coefficient of electrical resistivity, in K⁻¹ (i.e. per Kelvin).

The calculation unit 120 is then configured to determine a power loss PₒL as a function of the electrical intensity I_{cond} measured in the electrical conductor and the electrical resistance R_{c} of the electrical conductor that has been determined.

This power loss PₒL is notably determined as follows:

$$PoL = Rc \times {I}_{cond}^{2}$$

The calculation unit 120 is configured then to determine the annual power losses induced by the heating of the electrical conductor 10. The quantity of emissions of carbon dioxide is then determined as a function of these annual power losses.

To determine the quantity of emissions of CO₂ as a function of these annual power losses it is possible to use a scale factor multiplied by the annual power losses.

Generally speaking, the quantity of emissions of CO₂ as a function of these annual power losses can be determined utilising a relation defined by a distribution system operator.

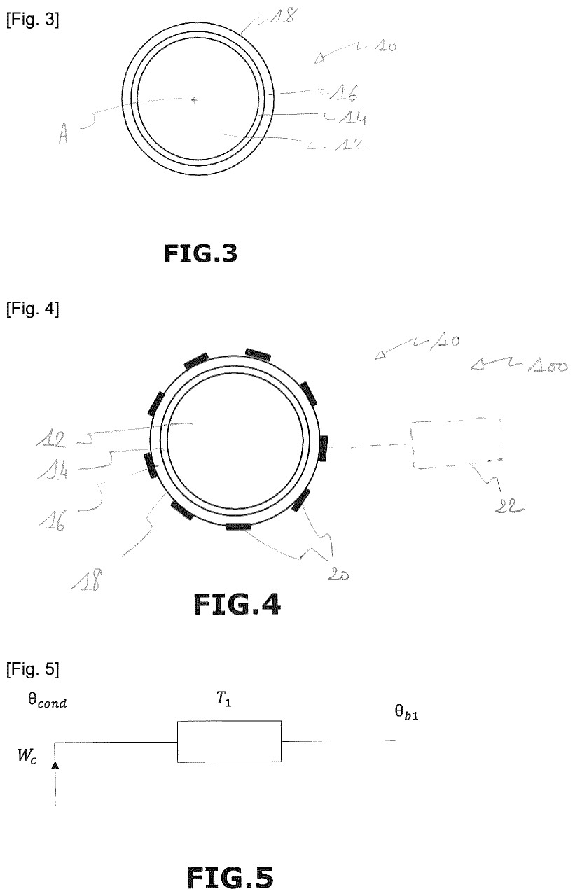

Referring to FIG. 3, an electrical cable 10 comprises an electrical conductor 12, a first layer 14 of material around the electrical conductor 12 and a second layer 16 of material around the first layer 14 of material.

The electrical conductor 12 extends along a longitudinal axis A.

The first layer 14 of material and the second layer 16 of material extend along the longitudinal axis A around the electrical conductor 12.

The first layer 14 of material is for example a layer formed of an electrically insulative material. The first layer 14 of material may therefore be considered as an insulating layer.

The second layer 16 of material here forms an external layer of the electrical cable 10. The second layer 16 of material forms an external surface 18 of the electrical cable 10.

The second layer 16 of material is for example an external sheath.

More generally, the electrical cable 10 may include one or more layers of material surrounding the electrical conductor 12. The electrical cable 10 may notably comprise one or more of the following: a screen, a semiconductor layer, an insulative layer, an external sheath.

In a preferred configuration the electrical cable 10 comprises around the conductor, in order of disposition from the centre to the periphery: a semiconductor layer, an insulative layer, a screen and an external sheath. This configuration corresponds for example to an electrical cable configured for a medium-voltage (between 1 and 52 kV) network.

In one embodiment the determination system 100 is preferably non-invasive and/or non-destructive. In other words, neither said at least one temperature sensor 20 nor the measurement device 112 is disposed inside the electrical cable.

In this embodiment the calculation unit 120 comprises a determination unit 22 configured to determine the conductor temperature Θ_{cond} by means of a physical model notably utilising a peripheral temperature Θ_{b1} measured by at least one temperature sensor 20 disposed on an external surface of the electrical cable 10.

The determination of this conductor temperature Θ_{cond} by means of this physical model is described hereinafter with reference to FIGS. 4 to 12.

As depicted in FIG. 4, the determination system 100 comprises said at least one temperature sensor 20 and a determination unit 22.

The determination unit 22 is part of the calculation unit 120.

The determination system 100 may comprise a plurality of temperature sensors 20 distributed around the longitudinal axis A in the same plane transverse to this longitudinal axis A. In the FIG. 4 example the determination system 100 comprises nine temperature sensors 20.

The temperature sensor or sensors 20 is or are configured to measure a peripheral temperature Θ_{b1}. In this configuration, in which the temperature sensors 20 are disposed at the level of the external surface 18 of the electrical cable 10, the peripheral temperature Θ_{b1} corresponds to the surface temperature of the electrical cable 10.

The temperature sensor or sensors 20 is or are connected to the determination unit 22 in such a manner as to communicate the peripheral temperature Θ_{b1} to this determination unit 22.

The temperature sensors 20 are preferably equally distributed around the longitudinal axis A. The determination system 100 may provide one or more temperature sensors 20 further distributed along the longitudinal axis A in such a manner as to measure the peripheral temperature Θ_{b1} at different locations along the electrical cable 10.

This determination unit 22 is configured to determine the conductor temperature Θ_{cond}, i.e. the temperature of the electrical conductor 12.

This determination is carried out in a non-invasive and non-destructive manner. Thus no component is inserted under the layers of material or in the proximity of the electrical conductor 12 to determine its conductor temperature Θ_{cond}. Furthermore, no layer of material of the electrical cable 10 is damaged or pierced to carry out this determination. No third-party component is integrated into the fabrication of the electrical cable 10 such as an optical fibre or a sensor in one of the layers of the electrical cable 10 or between these layers of material.

This enables determination of the conductor temperature in an existing electrical cable, for example already installed in situ, without the necessity to damage it or to insert any measuring tool in it.

It is considered here that the addition of additional layers of material or measuring instruments is not invasive or destructive.

The determination unit 22 utilises a physical model enabling the conductor temperature Θ_{cond} to be determined as a function of the peripheral temperature Θ_{b1} measured by the sensor or sensors 20.

Referring to FIG. 5, the diffusion of heat through the electrical cable 10 is represented in diagrammatic form to depict the physical model utilised by the determination unit 22.

This physical model is based on the fact that the diffusion of heat through the layers of an electrical cable follows a behaviour close to that of the circulation of a current in an electrical circuit including an electrical resistance.

The physical model therefore establishes a relation between the thermal resistance T₁ of said at least one layer of material. The thermal resistance T₁ may correspond to the thermal resistance of one or more of the layers of material. In the FIG. 4 example the thermal resistance T₁ represents the thermal resistance of the combination of the first layer 14 of material and the second layer 16 of material. In this physical model the first layer 14 of material and the second layer 16 of material therefore form one and the same layer of material having a layer thermal resistance denoted T₁.

The passage of the current inside the conductor generates heating inducing a thermal flux W_{c}.

In this physical model the voltage difference ΔU at the terminals of an electrical resistance is close to a temperature difference ΔΘ between the internal and external surfaces of said at least one layer of material (i.e. at the boundaries of this layer of material).

In the application of the electrical cable 10, the temperatures at the boundaries of the thermal resistance T₁ are on the one hand the conductor temperature Θ_{cond} and on the other hand the peripheral temperature Θ_{b1}. The temperature difference ΔΘ is therefore expressed as follows: ΔΘ=Θ_{cond}−Θ_{b1}.

In this physical model a mathematical relation is established between the thermal flux W_{c}, the thermal resistance T₁ and the temperature difference ΔΘ at the boundaries of this thermal resistance. This relation is as follows:

$$Δθ = {T}_{1} * {W}_{c}$$

with
ΔΘ being the temperature difference ΔΘ between the internal and external surfaces of said at least one layer of material,
T₁ being the thermal resistance of said at least one layer of material,
W_{c} being the thermal flux generated by the heating of the conductor.

The conductor temperature Θ_{cond} can therefore be expressed as follows:

$${θ}_{cond} = {θ}_{b 1} + {W}_{c} * {T}_{1}$$

with
Θ_{cond} being the conductor temperature,
Θ_{b1} being the peripheral temperature.

The thermal resistance T₁ of at least one layer of material is determined as follows:

$${T}_{1} = \frac{{ρ}_{T}}{2 \times π} ln ( 1 + 2 * \frac{{t}_{1}}{{d}_{c}} )$$

with
ρ_{T} being the thermal conductivity of said at least one layer of material,
d_{c} being the internal diameter of said at least one layer of material,
t₁ being the thickness of said at least one layer of material.

The physical model comprises two modes of determination of the conductor temperature Θ_{cond}.

In the first determination mode, the determination system 100 comprises a device for measuring the electrical intensity I_{cond} of an electrical current circulating in said at least one electrical conductor 12.

The measuring device is a non-invasive device, notably of the Rogowski coil type.

In the second determination mode, the determination system 100 comprises at least one additional layer 24 of material and at least one additional temperature sensor 26.

This second determination mode makes it possible to dispense with the use of the electrical intensity I_{cond} of the current circulating in the conductor.

Said at least an additional layer 24 of material is disposed around said at least one layer of material. The additional layer or layers 24 of material cover(s) said at least one temperature sensor 22, as can be seen in FIG. 6.

These two determination modes may be used for the determination of the conductor temperature Θ_{cond} using different models of an electrical cable 10. These different models may imply different hypotheses (e.g. dielectric losses taken into account or not) or different configurations of the electrical cable 10.

The determination unit 22 is configured to use the first and/or second determination mode(s). The determination unit 22 is configured to determine the conductor temperature Θ_{cond} using one or more models, notably one or more of the models described hereinafter.

Electrical Cable with No Screen and Dielectric Losses not Taken into Account

The determination unit 22 is configured to determine the conductor temperature Θ_{cond} according to first and second models respectively depicted in FIGS. 5 and 7.

To be more specific, the determination unit 22 is configured for the determination of the conductor temperature Θ_{cond} in the first model by means of the first determination mode. The determination unit 22 is configured for the determination of the conductor temperature Θ_{cond} in the second model by means of the second determination mode.

In the first and second models the dielectric losses in said at least one layer of material are not taken into account or are considered to be minimal.

In these first and second models the electrical cable 10 has no screen.

The first model applies to the electrical cable 10 comprising an electrical conductor 12 and one or more layers of material surrounding the electrical conductor 12. One or more temperature sensors 20 is or are disposed on the external surface 18 of said at least one layer of material.

The electrical cable 10 in FIG. 4 is an example compatible with this first model.

As indicated above, the conductor temperature Θ_{cond} may be expressed as follows:

$${θ}_{cond} = {θ}_{b 1} + {W}_{c} * {T}_{1}$$

with
Θ_{cond} being the conductor temperature,
Θ_{b1} being the peripheral temperature,
T₁ being the thermal resistance of said at least one layer of material,
W_{c} being the thermal flux generated by the heating of the conductor.

In the first determination mode the thermal flux W_{c} due to the heating of the electrical conductor 12 is expressed as follows:

$${W}_{c} = {R}_{c} * {I}_{cond}^{2}$$

R_{c} being the electrical resistance of the electrical conductor,
I_{cond} being the intensity of the current circulating in the electrical conductor.

The conductor temperature Θ_{cond} in the first determination mode, i.e. as a function of the intensity of the electrical conductor, is therefore expressed as follows:

$${θ}_{cond} = {θ}_{b 1} + {R}_{c} * {I}_{cond}^{2} * {T}_{1}$$

As seen above, the electrical resistance R_{c} of the electrical conductor is expressed as follows:

$${R}_{c} = {R}_{0} \times ( 1 + {α}_{20} \times ( {θ}_{cond} - 20 ) ) \times ( 1 + {y}_{s} + {y}_{p} )$$

with
R₀ being the direct current resistance of the conductor at 20° C., in Ohms,
Yₛ being the skin effect factor, no units,
Yₚ being the proximity effect factor, no units,
α₂₀ being the coefficient of electrical resistivity, in K⁻¹ (i.e. per Kelvin). The parameters R₀, Yₛ, Yₚ and α₂₀ are values linked to the structure and to the nature of the electrical conductor.

The conductor temperature Θ_{cond} can therefore be expressed as follows:

$${θ}_{cond} = \frac{{θ}_{b 1} - {R}_{0}^{2} \times {T}_{1} \times ( 1 - 20 \times {α}_{20} )}{1 - {R}_{0} \times {I}_{cond}^{2} \times {α}_{20} \times {T}_{1}}$$

In the second determination mode, i.e. without the intensity I_{cond} of the conductor, the determination system 100 comprises an additional structure depicted in FIG. 6. The determination system 100 therefore comprises at least one additional layer 24 of material and at least one additional temperature sensor 26.

Said at least one additional layer 24 of material has an additional thermal resistance T_{b}.

Said at least one additional layer 24 of material is for example at least one electrically insulative layer.

The material of said at least one additional layer 24 of material preferably has a thermal resistance between 0.001 m²·K/W and 0.1 m²·K/W. In this range of thermal resistance said at least one layer 24 of material makes it possible to avoid overheating of the conductor while allowing a temperature difference that is sufficiently large to be measured.

Said at least one additional temperature sensor 26 enables measurement of an additional peripheral temperature Θ_{b2} at the level of this external surface 28 of said at least one additional layer 24 of material.

The determination system 100 may comprise a plurality of additional temperature sensors 26 distributed around the longitudinal axis A in the same plane transverse to the longitudinal axis A. In the FIG. 6 example the determination system 100 comprises nine additional temperature sensors 26.

The additional temperature sensor or sensors 26 is or are connected to the determination unit 22 in such a manner as to communicate the additional peripheral temperature Θ_{b2} to this determination unit 22.

The additional temperature sensors 26 are preferably equally distributed around the longitudinal axis A. The determination system 100 can provide one or more additional temperature sensors 26 further distributed along the longitudinal axis A in such a manner as to measure the additional peripheral temperature Θ_{b2} at different locations along the electrical cable 10.

The number and/or the angular position and/or the longitudinal position of the temperature sensors 20 is or are respectively identical to the number and/or the angular position and/or the longitudinal position of the additional temperature sensors 26.

The electrical cable 10 equipped with said at least one additional layer 24 of material and said at least one additional temperature sensor 26 is modelled by a second model in FIG. 7. Said at least one additional layer 24 of material is considered a resistance of value T_{b} in series with the resistance of value T₁ corresponding to said at least one layer of material.

In this second determination mode the thermal flux W_{c} is expressed as follows:

$${W}_{c} = \frac{( {θ}_{b 1} - {θ}_{b 2} )}{{T}_{b}}$$

The conductor temperature can therefore be expressed as follows:

$${θ}_{cond} = {θ}_{b 1} + \frac{( {θ}_{b 1} - {θ}_{b 2} )}{{T}_{b}} \times {T}_{1}$$

The conductor temperature Θ_{cond} can therefore be determined without needing the value of the intensity of the current circulating in the electrical conductor 12. This determination is rendered possible by the addition of an additional layer and an additional sensor.

Electrical Cable with No Screen and with Dielectric Losses Taken into Account

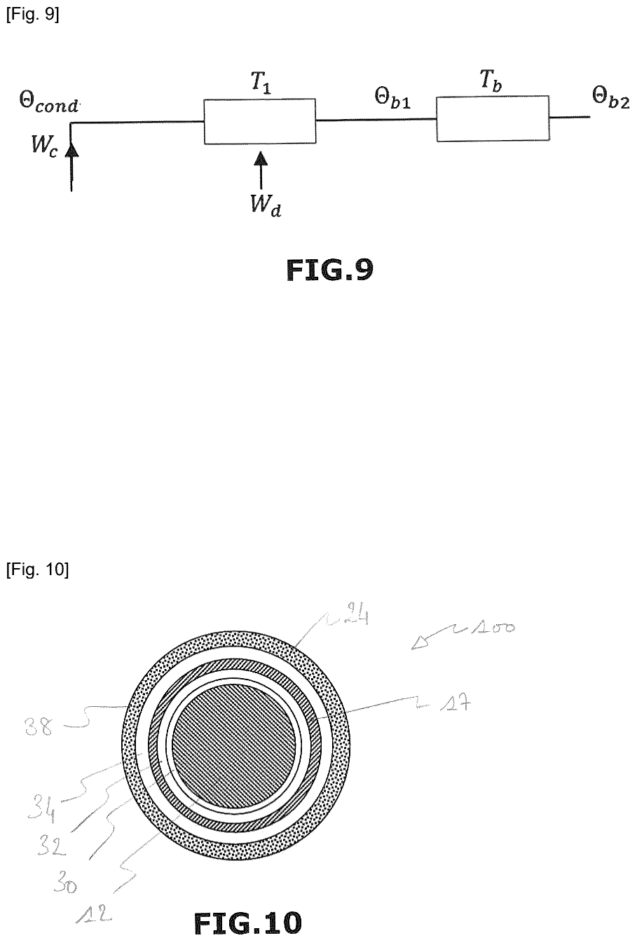

The determination unit 22 is configured to determine the conductor temperature Θ_{cond} using third and fourth models respectively depicted in FIGS. 8 and 9.

To be more specific, the determination unit 22 is configured to determine the conductor temperature Θ_{cond} using a third model by means of the first determination mode. The determination unit 22 is configured for the determination of the conductor temperature Θ_{cond} in the fourth model by means of the second determination mode.

In the third and fourth models the dielectric losses in said at least one layer of material are taken into account.

In the third and fourth models the electrical cable 10 has no screen.

In the third and fourth models the dielectric losses in said at least one layer of material are considered as a loss thermal flux W_{d} at the level of the resistance of value T₁ corresponding to said at least one layer of material. This loss thermal flux W_{d} can be seen FIGS. 8 and 9.

The third model applies to the electrical cable 10 comprising an electrical conductor 12 and one or more layers of material surrounding the electrical conductor 12. One or more temperature sensors 20 is or are disposed on the external surface 18 of said at least one layer of material.

The electrical cable 10 in FIG. 4 is an example compatible with this third model.

In the first determination mode the conductor temperature Θ_{cond} may be expressed as follows as a function of the electrical intensity I_{cond}:

$${θ}_{cond} = {θ}_{b 1} + {T}_{1} \times ( {W}_{c} - \frac{{W}_{d}}{2} )$$

This conductor temperature Θ_{cond} may equally be expressed as follows by expanding R_{c} as described above:

$${θ}_{cond} = \frac{{θ}_{b 1} - ( {R}_{0} {I}_{cond}^{2} ) {T}_{1} ( 1 - 20 \times {α}_{20} ) + \frac{1}{2} {T}_{1} {W}_{d}}{1 - {R}_{0} {I}_{cond}^{2} {α}_{20} {T}_{1}}$$

In the second determination mode, i.e. without the conductor intensity I_{cond}, the determination system 100 comprises an additional structure as depicted in FIG. 6. The determination system 100 therefore comprises at least one additional layer 24 of material and at least one additional temperature sensor 26.

The electrical cable 10 equipped with said at least one additional layer 24 of material and said at least one additional temperature sensor 26 is modelled by a fourth model in FIG. 9.

Said at least one additional layer 24 of material is considered as a resistance of value T_{b} in series with the resistance of value T₁ corresponding to said at least one layer of material.

In this second determination mode the conductor temperature Θ_{cond} is expressed as follows:

$${θ}_{cond} = {θ}_{b 1} + {T}_{1} \times ( \frac{( {θ}_{b 1} - {θ}_{b 2} )}{{T}_{b}} - \frac{{W}_{d}}{2} )$$

The above equation is obtained by considering the following equations:

$${θ}_{cond} = {θ}_{b 1} + {T}_{1} \times ( {W}_{c} + \frac{{W}_{d}}{2} )$$

$$and$$

$${θ}_{b 1} = {θ}_{b 2} + {T}_{b} \times ( {W}_{d} + {W}_{c} )$$

The loss thermal flux W_{d} is determined as a function of the voltage applied to the electrical conductor 12, the frequency of the voltage applied to the electrical conductor 12 and the dielectric characteristics of said at least one layer of material.

Electrical Cable with Screen and with Dielectric Losses Taken into Account

The determination unit 22 is further configured to determine the conductor temperature Θ_{cond} in a configuration of the electrical cable 10 comprising a screen 17.

As depicted in FIG. 10, the electrical cable 10 comprises an electrical conductor 12, one or more layers of material surrounding the electrical conductor 12 and a screen 17 surrounding said layers of material.

Said layers of material are for example a dielectric layer 30 surrounding the electrical conductor 12 and an insulative layer 32 disposed around the dielectric layer 30 and the screen 17.

The electrical cable 10 also comprises an external layer 34, for example an external sheath, defining an external surface 38 of the external layer 34. The external layer 34 may comprise a plurality of layers of material.

The external layer 34 has a thermal resistance T₃.

One or more temperature sensors 20 is or are disposed on the external surface 38 of the external layer 34.

Losses in the screen 17 are modelled by a screen thermal flux Wₛ. These losses are due to heating by the Joule effect in the screen 17.

Determination of the screen thermal flux Wₛ requires an invasive measurement of the electrical cable 10. To avoid having to express the conductor temperature Θ_{cond} as a function of the thermal flux Wₛ, it is proposed here to combine the first and second determination modes encountered above. In other words, there is provision here for expressing the conductor temperature Θ_{cond} as a function of the intensity I_{cond} of the current circulating in the electrical conductor 12 and to utilise an additional structure comprising at least one additional layer 24 of material and at least one additional temperature sensor 26, as can be seen in FIG. 10.

Said at least one additional layer 24 of material has a thermal resistance T_{b}.

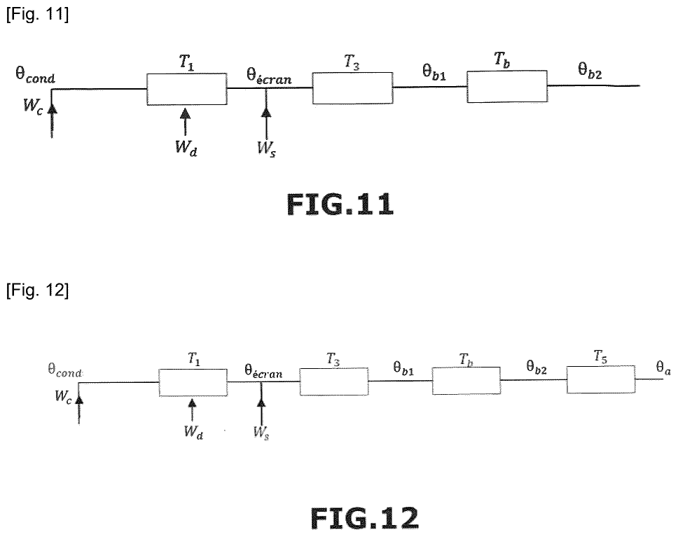

A fifth model is depicted in FIG. 11 taking into account the screen losses (thermal flux Wₛ) and the dielectric losses in said at least one layer (thermal flux W_{d}) and comprising three resistances in series to model the thermal resistances of said at least one layer of material (T₁), of the external sheath 34 (T₃) and of said at least one additional layer 24 of material (T_{b}).

In this fifth model the conductor temperature Θ_{cond} is expressed as follows:

$${θ}_{cond} = \frac{{θ}_{b 1} + {T}_{3} \frac{Δ {θ}_{b}}{{T}_{b}} + \frac{1}{2} {T}_{I} {W}_{d} - ( {R}_{0} {i}^{2} ) {T}_{1} ( 1 - 20 \times {α}_{20} )}{1 - {R}_{0} {I}_{cond}^{2} {α}_{20} {T}_{1}}$$

Θ_{b1} being the peripheral temperature measured by said at least one temperature sensor 20,
Θ_{b2} being the additional peripheral temperature measured by said at least one additional temperature sensor 26,
ΔΘ being the temperature difference Θ_{b1}−Θ_{b2},
T₁ being the thermal resistance of said at least one layer of material,
T_{b} being the thermal resistance of said at least one additional layer 24 of material,
T₃ being the thermal resistance of the external layer 34,
R₀ being the direct current resistance of the conductor at 20° C., expressed in Ohms,
Yₛ being the skin effect factor, no units,
Yₚ being the proximity effect factor, no units,
α₂₀ being the coefficient of electrical resistivity, expressed in K⁻¹ (i.e. per Kelvin).

The determination unit 22 is therefore capable of determining the conductor temperature Θ_{cond} independently of the screen thermal flux Wₛ.

This expression of the conductor temperature Θ_{cond} is obtained considering that:

$$\begin{matrix}{θ}_{cond} = {θ}_{surf} + n ( {W}_{c} + {W}_{s} + {W}_{d} ) {T}_{3} + ( {W}_{c} + \frac{{W}_{d}}{2} ) {T}_{1} \\ n ( {W}_{c} + {W}_{s} + {W}_{d} ) = \frac{Δ {θ}_{b}}{{T}_{b}} \\ {θ}_{c onducteur} = {θ}_{b 1} + {T}_{3} \frac{Δ {θ}_{b}}{{T}_{b}} + ( {W}_{c} + \frac{{W}_{d}}{2} ) {T}_{1}\end{matrix}$$

with
Θ_{surf} being the peripheral temperature at the level of the external surface of the electrical cable 10, and
n being the number of electrical conductors 12.

As described in detail above, the thermal resistance T₁ is determined as follows:

$${T}_{1} = \frac{{ρ}_{T}}{2 \times π} ln ( 1 + 2 * \frac{{t}_{1}}{{d}_{c}} )$$

The thermal resistance T₃ of the thermal layer 34 is determined as follows:

$${T}_{3} = \frac{{ρ}_{T}}{2 \times π} ln ( 1 + 2 * \frac{{t}_{3}}{{D}_{a}} )$$

with
t₃ being the thickness of the external layer 34,
Dₐ being the inside diameter of the external layer 34.

In a sixth model depicted in FIG. 12, the determination unit is also configured to determine the conductor temperature Θ_{cond} in a manner independent of the surrounding environment, notably the temperature in that surrounding environment.

This sixth model applies to the same configuration of the electrical cable 10 as the fifth model. In other words, the sixth model applies to an electrical cable of the FIG. 10 type with an additional structure and a screen 17.

Depending on the surrounding environment, heat will be evacuated less or more from the electrical cable 10. The surrounding environment is modelled by a layer of material with a certain thermal resistance T₅ and a temperature θₐ corresponding to the ambient temperature of the surrounding environment.

The conductor temperature Θ_{cond} may be expressed as follows:

$${θ}_{cond} = {θ}_{a} + ( {W}_{c} + \frac{{W}_{d}}{2} ) {T}_{1} + n ( {W}_{c} + {W}_{d} + {W}_{S} ) {T}_{3} + n ( {W}_{c} + {W}_{d} + {W}_{s} ) {T}_{b} + n ( {W}_{c} + {W}_{d} + {W}_{S} ) {T}_{5}$$

The temperature difference Θ_{b1}−Θ_{b2} on either side of the additional layer 24 of material enables the conductor temperature Θ_{cond} to be expressed as follows:

$${θ}_{cond} = {θ}_{b 1} + ( {W}_{c} + \frac{{W}_{d}}{2} ) {T}_{1} + n ( {W}_{c} + {W}_{d} + {W}_{s} ) {T}_{b}$$

$$and$$

$${θ}_{b 1} - {θ}_{b 2} = n ( {W}_{c} + {W}_{d} + {W}_{s} ) {T}_{b}$$

The conductor temperature Θ_{cond} can therefore be determined by the determination unit 22 independently of the surrounding environment.

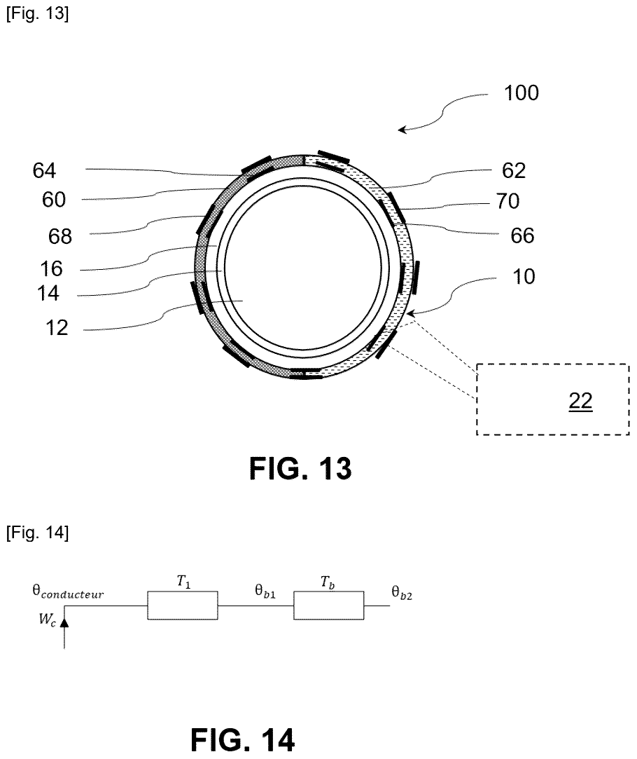

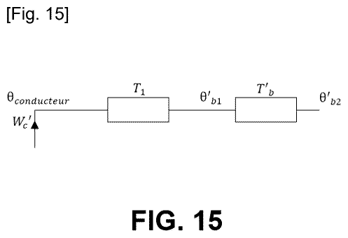

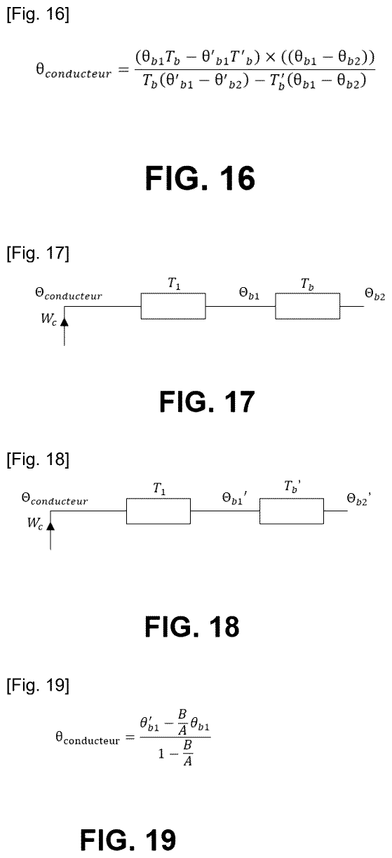

Referring to FIGS. 13 to 19, the determination unit 22 is further configured to determine the conductor temperature Θ_{cond} in a seventh model and an eighth model.

In the seventh and eighth models the determination unit 22 is configured to determine the conductor temperature Θ_{cond} without necessitating determination of the thermal flux W_{c} generated by the circulation of an electrical current in the electrical conductor.

The determination system 100 for these seventh and eighth models is similar to that in FIG. 6 with the difference that said at least one additional layer 24 comprises a first additional layer portion 60 and a second additional layer portion 62, as depicted in FIG. 13.

The first additional layer portion 60 has a first additional thermal resistance T_{b}. The second additional layer portion 62 has a second additional thermal resistance T′_{b}. The first additional thermal resistance T_{b} and the second additional thermal resistance T′_{b} are different.

This difference between the first additional thermal resistance T_{b} and the second additional thermal resistance T′_{b} can be obtained by utilisation of a different material and/or of one or more geometrical characteristics that differ between the first additional layer portion 60 and the second additional layer portion 62. One example of a different geometrical characteristic is a different thickness along an axis perpendicular to the longitudinal axis A along which the electrical conductor extends.

This difference between the first additional thermal resistance T_{b} and the second additional thermal resistance T′_{b} enables construction of two different equations having for unknown the conductor temperature Θ_{cond}. It is therefore possible to dispense with knowing the thermal flux W_{c}.

A plurality of first temperature sensors 64 are disposed on the external surface 18 of said at least one layer of material, between said external surface 18 and the first additional layer portion 60. The plurality of first temperature sensors 64 enable measurement of a first peripheral temperature Θ_{b1} at the level of the external surface 18 of said at least one layer of material.

A plurality of second temperature sensors 66 are disposed on the external surface 18 of said at least one layer of material, between said external surface 18 and the second additional layer portion 62. The plurality of second temperature sensors 66 enable measurement of a second peripheral temperature Θ′_{b1} at the level of the external surface 18 of said at least one layer of material.

A plurality of first additional temperature sensors 68 are disposed on an external surface of the first additional layer portion 60 to measure a first additional peripheral temperature Θ_{b2}.

A plurality of second additional temperature sensors are disposed on an external surface of the second additional layer portion to measure a second additional peripheral temperature Θ′_{b2}.

The determination unit 22 is configured to determine the conductor temperature Θ_{cond} as a further function of the first additional thermal resistance T_{b} and the second additional thermal resistance T′_{b} and the first additional peripheral temperature Θ_{b2} and the second additional peripheral temperature Θ′_{b2}.

Electrical Cable with No Screen without Dielectric Losses Taken into Account

The seventh model is depicted in FIGS. 14 to 16.

In the seventh model the conductor temperature Θ_{cond} is expressed as follows:

$${θ}_{c onducteur} = \frac{( {θ}_{b 1} {T}_{b} - {θ}_{b 1}^{′} {T}_{b}^{′} ) \times ( ( {θ}_{b 1} - {θ}_{b 2} ) )}{{T}_{b} ( {θ}_{b 1}^{′} - {θ}_{b 2}^{′} ) - {T}_{b}^{′} ( {θ}_{b 1} - {θ}_{b 2} )}$$

This equation is obtained via the following expansions:

$$\begin{matrix}\frac{( {θ}_{c onducteur} - {θ}_{b 1} )}{{T}_{1}} = \frac{( {θ}_{b 1} - {θ}_{b 2} )}{{T}_{b}} \\ \frac{( {θ}_{c onducteur} - {θ}_{b 1}^{′} )}{{T}_{1}} = \frac{( {θ}_{b 1}^{′} - {θ}_{b 2}^{′} )}{{T}_{b}^{l}} \\ ( {θ}_{c onducteur} - {θ}_{b 1} ) \frac{{T}_{b}}{( {θ}_{b 1} - {θ}_{b 2} )} = ( {θ}_{c onducteur} - {θ}_{b 1}^{′} ) \frac{{T}_{b}^{′}}{( {θ}_{b 1}^{′} - {θ}_{b 2}^{′} )} \\ {θ}_{c onducteur} = \frac{{θ}_{b 1} {T}_{b} - {θ}_{b 1}^{′} {T}_{b}^{′}}{( {θ}_{b 1}^{′} - {θ}_{b 2}^{′} )} / ( \frac{{T}_{b}}{( {θ}_{b 1} - {θ}_{b 2} )} - \frac{{T}_{b}^{′}}{( {θ}_{b 1}^{′} - {θ}_{b 2}^{′} )} )\end{matrix}$$

Electrical Cable with No Screen with Dielectric Losses Taken into Account

The eighth model is depicted in FIGS. 17 to 19.

In the eighth model, the conductor temperature Θ_{cond} is expressed as follows:

$${θ}_{conducteur} = \frac{{θ}_{b 1}^{′} - \frac{B}{A} {θ}_{b 1}}{1 - \frac{B}{A}}$$

This equation is obtained via the following expansions:

$$\begin{matrix}{θ}_{conducteur} = {θ}_{b 1} + {T}_{1} \times ( \frac{( {θ}_{b 1} - {θ}_{b 2} )}{{T}_{b}} - \frac{{W}_{d}}{2} ) \\ {θ}_{conducteur} = {θ}_{b 1}^{′} + {T}_{1} \times ( \frac{( {θ}_{b 1}^{′} - {θ}_{{b2}^{′}} )}{{T}_{b}^{′}} - \frac{{W}_{d}}{2} ) \\ A = ( \frac{( {θ}_{b 1} - {θ}_{b 2} )}{{T}_{b}} - \frac{{W}_{d}}{2} ) \\ B = ( \frac{( {θ}_{b 1}^{′} - {θ}_{b 2}^{′} )}{{T}_{b}^{′}} - \frac{{W}_{d}}{2} )\end{matrix}$$

Θ_{b1}′ being the first peripheral temperature at the level of the external surface 18 of said at least one layer of material,
Θ_{b2}′ being the second additional peripheral temperature,
T_{b}′ being the second additional thermal resistance.

1. A determination system for determination of a quantity of emissions of carbon dioxide resulting from the heating of an electrical conductor of an electrical cable by the Joule effect, said determination system comprising:
an electrical cable comprising at least one electrical conductor and at least one layer of material surrounding said at least one conductor,
a measurement unit associated with the electrical cable, said measurement unit comprising at least one temperature sensor and a device for measurement of the electrical intensity I_{cond} of an electrical current circulating in the electrical conductor,
a calculation unit configured to communicate information with the measurement unit, the calculation unit being configured to determine the conductor temperature Θ_{cond} by means of said at least one temperature sensor, said calculation unit being further configured to determine a quantity of emissions of carbon dioxide resulting from the heating of the electrical conductor by the Joule effect as a function of the conductor temperature Θ_{cond} and the electrical intensity I_{cond} in the electrical conductor.

2. The determination system according to claim 1, in which the calculation unit is configured to determine an electrical resistance R_{c} of the electrical conductor as a function of the conductor temperature Θ_{cond}.

3. The determination system according to claim 2, in which the calculation unit is configured to determine a power loss PₒL as a function of the electrical intensity I_{cond} in the electrical conductor and the electrical resistance R_{c} of the electrical conductor, the calculation unit being configured to determine the quantity of emissions of carbon dioxide as a function of said power loss PₒL.

4. The determination system according to claim 1, further comprising a measurement module mounted on the electrical cable, said measurement module comprising at least one of the following: said at least one temperature sensor and the device for measurement of the electrical intensity I_{cond}.

5. The determination system according to claim 4, in which the measurement module further comprising the calculation unit.

6. The determination system according to claim 4, in which the measurement module is configured to be removably mounted on the electrical cable.

7. The determination system according to claim 6, in which the measurement module comprises a device for fixing it to the electrical cable.

8. The determination system according to claim 1, in which said at least one temperature sensor is disposed on an external surface of said at least one layer of material to measure a peripheral temperature Θ_{b1} at the level of the external surface of said at least one layer of material, the calculation unit being configured to determine the conductor temperature Θ_{cond} as a function of the peripheral temperature Θ_{b1}.

9. The determination system according to claim 8, further comprising:
an additional layer of material disposed around said at least one layer of material and covering said at least one temperature sensor,
at least one additional temperature sensor disposed on an external surface of said additional layer of material to measure an additional peripheral temperature Θ_{b2} at the level of the external surface of said at least one additional layer of material.

10. The determination system according to claim 9, in which the calculation unit is configured to determine the conductor temperature Θ_{cond} as a function of the peripheral temperature Θ_{b1} and the additional peripheral temperature Θ_{b2}.

11. The determination system according to claim 9, in which said at least one additional layer of material comprises:
a first additional layer portion having a first additional thermal resistance T_{b}, and
a second additional layer portion having a second additional thermal resistance T′_{b}, the first additional thermal resistance T_{b} and the second additional thermal resistance T′_{b} being different,
and in which said at least one additional temperature sensor comprises:
at least one first additional temperature sensor disposed on an external surface of the first additional layer portion to measure a first additional peripheral temperature Θ_{b2},
at least one second additional temperature sensor disposed on an external surface of the second additional layer portion to measure a second additional peripheral temperature Θ′_{b2},
the determination unit being configured to determine the conductor temperature Θ_{cond} further as a function of the first additional thermal resistance T_{b} and the second additional thermal resistance T′_{b} and the first additional peripheral temperature Θ_{b2} and the second additional peripheral temperature Θ′_{b2}.

12. The determination system according to claim 11, in which said at least one first additional layer and said at least one second additional layer are disposed on different angular sectors around the electrical conductor.

13. The determination system according to claim 12, in which said at least one temperature sensor comprises:
at least one first sensor disposed on the external surface of said at least one layer of material, between said external surface and the first additional layer portion, to measure a peripheral temperature Θ_{b1} at the level of the external surface of said at least one layer of material,
at least one second sensor disposed on the external surface of said at least one layer of material, between said external surface and the second additional layer portion, to measure a peripheral temperature Θ′_{b1} at the level of the external surface of said at least one layer of material.

14. The determination system according to claim 13, in which the determination unit is configured to determine the conductor temperature Θ_{cond} on the basis of the following equation:
$${θ}_{conducteur} = \frac{( {θ}_{b 1} {T}_{b} - {θ}_{b 1}^{′} {T}_{b}^{′} ) \times ( ( {θ}_{b 1} - {θ}_{b 2} ) )}{{T}_{b} ( {θ}_{b 1}^{′} - {θ}_{b 2}^{′} ) - {T}_{b}^{′} ( {θ}_{b 1} - {θ}_{b 2} )}$$
T₁ being the thermal resistance of said at least one layer of material,
T_{b} being the first additional thermal resistance of the first additional layer of material,
T′_{b} being the second additional thermal resistance of the second additional layer of material,
Θ_{b1} is the peripheral temperature measured by said at least one first temperature sensor,
Θ_{b2} is the additional peripheral temperature measured by said at least one first additional temperature sensor,
Θ′_{b1} is the peripheral temperature measured by said at least one second temperature sensor,
Θ′_{b2} is the additional peripheral temperature measured by said at least one second additional temperature sensor.

15. The determination system according to claim 11, in which the first and second additional layer portions are made of different materials.

16. The determination system according to claim 11, in which the first and second additional layer portions have different thicknesses perpendicular to a longitudinal axis along which the electrical cable extends.
